(19) United States
(12) Patent Application Publication
(10) Pub. No.: US 2020/0390461 A1
(43) Pub. Date: Dec. 17, 2020

(54) FORCEPS WITH POWERED CUTTING DEVICE

(71) Applicant: Chris Geiger, Castle Rock, CO (US)

(72) Inventor: Chris Geiger, Castle Rock, CO (US)

(21) Appl. No.: 16/438,598

(22) Filed: Jun. 12, 2019

(51) Int. Cl. A61B 17/285; A61B 17/30; A61B 17/32

(52) U.S. Cl. CPC A61B 17/285; A61B 2017/00398; A61B 17/32; A61B 17/30

(57) ABSTRACT

An apparatus includes a first arm having a first gripping end and a first handle end, and a second arm having a second gripping end and a second handle end. The first handle end and the second handle end are joined so as to form a handle. A cutting element is disposed on the first arm, proximate the first gripping end, and an electronic circuit at least partially disposed in the first arm. The electronic circuit is connected to the cutting element, and the electronic circuit is selectively activatable so as to deliver an electric signal to the cutting element.

### INTRODUCTION

Forceps, tweezers, tongs, and the like are used in medical, hobby, utility, and other applications to aid in gripping and pulling objects, particularly small structures. For example, forceps can be used in medical procedures to aid in the attachment or removal of sutures. After suture attachment, instruments such as scissors are then used to cut the suture to the proper length. A similar procedure is used in suture removal, where the suture is first cut by a pair of scissors, then pulled with a pair of forceps. Both of these procedures require the use of two instruments, either simultaneously in two hands or sequentially in a single hand.

### SUMMARY

In one aspect, the technology relates to an apparatus including: a first arm having a first gripping end and a first handle end; a second arm having a second gripping end and a second handle end, wherein the first handle end and the second handle end are joined so as to form a handle; a cutting element disposed on the first arm, proximate the first gripping end; and an electronic circuit at least partially disposed in the first arm, wherein the electronic circuit is connected to the cutting element, and wherein the electronic circuit is selectively activatable so as to deliver an electric signal to the cutting element.

In an example, a switch connected to the electronic circuit, the switch selectively activates the electronic circuit. In another example, the switch includes a first contact on the first arm and a second contact on the second arm, when the first arm is in a first position, the switch is in a non-activated configuration, and when the first arm is in a second position, the switch is in an activated configuration. In yet another example, when in the first position, the first gripping end and the second gripping end are not in contact, and when in the second position, the first gripping end and the second gripping end are not in contact. In still another example, when the first arm is in a third position, the switch is in a non-actuated position and the first gripping end and the second gripping end are in contact.

In an example, the second position is disposed between the first position and the third position. In another example, a battery is disposed in the handle. In yet another example, the first gripping end is detachably engaged with the first handle end, and the second gripping end is detachably engaged with the second handle end.

In another aspect, the technology relates to an apparatus including: a forceps; and a cutting element connected to at least one arm of the forceps, wherein the cutting element is selectively activatable based on a position of the forceps.

In an example, a contact is disposed on each arm of the forceps; and an electronic circuit disposed in the forceps, the electronic circuit is connected to the cutting element and the contacts. In another example, when the forceps are in a first position, the electronic circuit is open. In yet another example, when the forceps are in a second position, the electronic circuit is closed. In still another example, when the forceps are in a third position, the electronic circuit is open, and the second position is between the first position and the third position.

In an example, a rechargeable battery is in communication with the electronic circuit. In another example, the at least one arm is disconnectable from the forceps.

In another aspect, the technology relates to an apparatus including: a forceps having a first arm, a second arm, and a handle portion connecting the first arm and the second arm, wherein each of the first arm and the second arm terminates at a connector element; a first gripping element configured to be releasably connected to the first arm connector element; a second gripping element configured to be releasably connected to the second arm connector element; an electronic circuit at least partially disposed in the first arm; and a cutting element disposed on the first gripping element, wherein the cutting element is configured to electronically engage with the electronic circuit.

In an example, a control switch is operatively coupled to the electronic circuit. In another example, the control switch includes a first contact on at least one of the first arm and the first gripping element and a second contact on at least one of the second arm and the second gripping element. In yet another example, at least one of a battery and a power cord are connected to the electronic circuit. In still another example, the first connector arm includes a cutting element contact in communication with the electronic circuit.

### BRIEF DESCRIPTION OF THE DRAWINGS

There are shown in the drawings, examples which are presently preferred, it being understood, however, that the technology is not limited to the precise arrangements and instrumentalities shown.

FIG. 1 depicts a perspective view of a gripping instrument with an integral cutting element.

FIG. 2 depicts a partial exploded perspective view of the gripping instrument of FIG. 1.

FIGS. 3A-3D are schematic diagrams of electronic circuits utilized in gripping instruments.

FIGS. 4A-4C are enlarged schematic views a gripping instrument in a plurality of positions.

FIGS. 5-6 are other examples of gripping instruments with integral cutting elements.

### DETAILED DESCRIPTION

In general, forceps, tweezers, tongs, or other gripping instruments have a pair of arms that are used to grip an object, usually by application of a squeezing force on the instrument (depending on instrument configuration). Certain of these instruments may also be locked in one or more positions. When in an open position, the arms of the instrument are spread away from each other, in examples by a spring or return force inherent in the material used to manufacture the instrument. Other types of instruments (e.g., forceps such as Pean, Gross-Maier, etc.) include a pivoting connection joining the two arms. Thumb and finger holes at the ends of these arms allow the gripping ends of the arms to be moved further apart and closer together. In the case of instruments having a pivoting connection, the thumb and finger holes allow those instruments to operate much like a pair of scissors. However, when the arms of such instruments are brought into a gripping position (with the tips close together, usually with an object present therebetween), the gripping portions merely grip the object without cutting or slicing as is the case with scissors.

The blades utilized on scissor-type instruments are often not sufficiently sharp to cut polypropylene sutures used in medical applications. As such, the patient having sutures removed therefrom is often subject to uncomfortable pushing and pinching which can make suture removal unpleasant. The technologies described herein incorporate a heating element that can be selectively activated so as to quickly cut the polypropylene sutures by melting the suture. The forceps can then be used to grip the cut suture. As such, the forceps with integrated cutting devices described herein may be used to more quickly remove sutures with a single instrument, without first cutting the suture with a pair of scissors, then removing the suture with a pair of forceps. As such, a user may utilize both functions of the cutting instrument-integrated forceps, without having to trade instruments, or utilize a cutting implement disposed, e.g., on an end of the instrument opposite the gripping portion. The integrated cutting instrument technologies described herein may be applied to forceps, tweezers, tongs, or other gripping instruments that are used to enable or supplement the gripping of objects, especially very fine or small objects. For clarity herein, however, the technologies will be described generally in the context of medical forceps used for the insertion and removal of sutures.

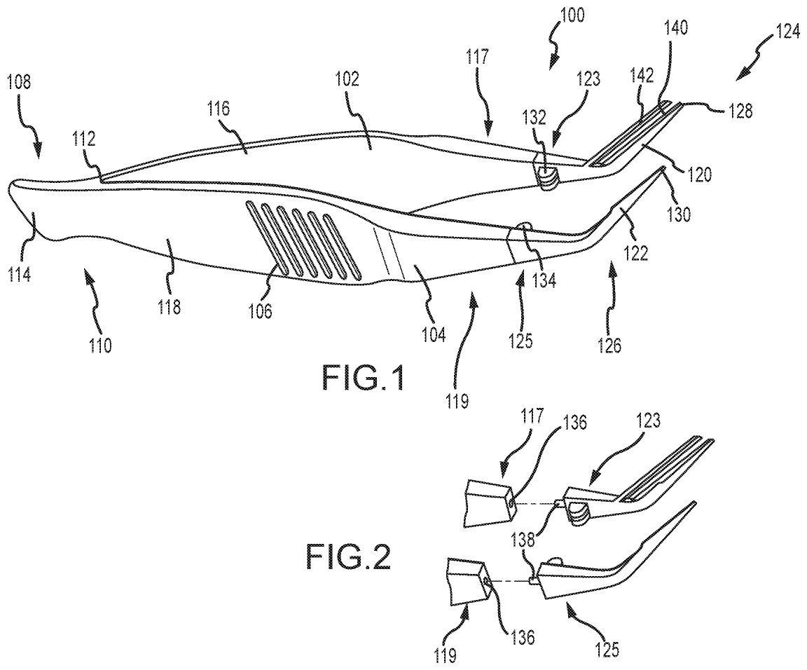

FIG. 1 is a perspective view of a gripping instrument, in this case, a pair of forceps 100. FIG. 2 is a partial exploded perspective view of the forceps 100 and is described simultaneously with FIG. 1. The forceps 100 include two arms 102, 104, each including on an exterior thereof one or more raised serrations, ribs, or ridges 106 to improve gripping of the forceps 100. The arms 102, 104 are connected at first ends 108, 110 thereof to form a joint 112 so as to form a handle 114. Typically, during use, the handle 114 of the forceps 100 may rest in or be cupped by the palm of the hand, while one finger and the thumb may apply opposing forces to the arms 102, 104 (whether the thumb or finger applies force to arm 102 or arm 104 is a function of how, and in which hand, the forceps 100 are held).

The arms 102, 104 each include a main portion 116, 118 (typically where the forceps 100 are gripped by a user) and a tip or gripping portion 120, 122 (typically where the forceps 100 capture an object). Terminal ends 123, 125 of each gripping portion 120, 122 are configured to connect to terminal ends 117, 119 of each main portion 116, 118. Second ends 124, 126 of each gripping portion 120, 122 are located distal from the terminal ends 123, 125 and each include a tip or gripping end 128, 130. Since the gripping portions 120, 122 can be disconnected from the main portions 116, 118, the gripping portions 120, 122 can be disposed of after use, or if they become damaged. In general, the tips 128, 130 may be the distal ends of the tip portions 120, 122 that contact each other when the two arms 102, 104 are forced together (without an object therebetween). Textured surfaces may be disposed thereon to reduce slipping of the object being gripped. Stops 132, 134 may prevent over-squeezing of the arms 102, 104 when the tips 128, 130 are in contact. The stops 132, 134 also define contacts that form a part of an electronic circuit for activating a cutting element, as described elsewhere herein.

The terminal ends 117, 119 include a recess 136 that is configured to receive a projection 138 that extends from the first ends 123, 125 of each gripping portion 120, 122. These recesses 136 and projections 138 are connector elements that mate to form a connector structure so as to secure the gripping portions 120, 122 to the main portions 116, 118 of the arms 102, 104. The connector structures should display sufficient robustness so as to allow the forceps 100 to be used to grip objects without disconnection. Other recess/projection configurations or connector structures are contemplated. Each connector element 136, 138 can include one or more contacts therein for completing the electronic circuit described below. At least one cutting element 140 is disposed on the forceps 100, more specifically, on the gripping portion 120 of the arm 102. The cutting element 140 may be disposed in a number of locations, but in the depicted example, the cutting element 140 extends along the gripping portion 120. The cutting element 140 is a resistive material formed of wire or metal that converts electricity into heat so as to become hot when activated. This heat is used to quickly melt or burn through polypropylene sutures or other structures. A guard 142 can be positioned so as to protect the cutting elements 140 from inadvertent contact with, e.g., skin, gauze, clothing, etc.

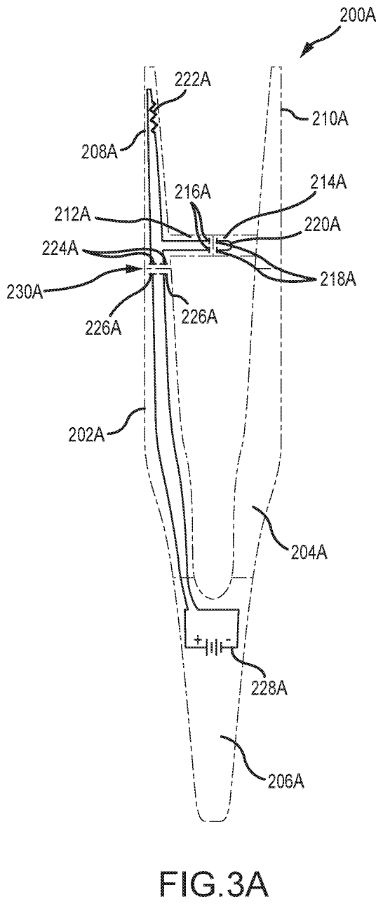

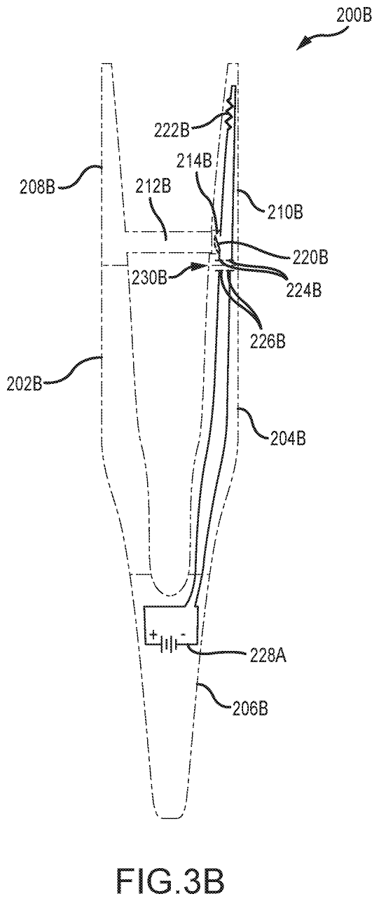

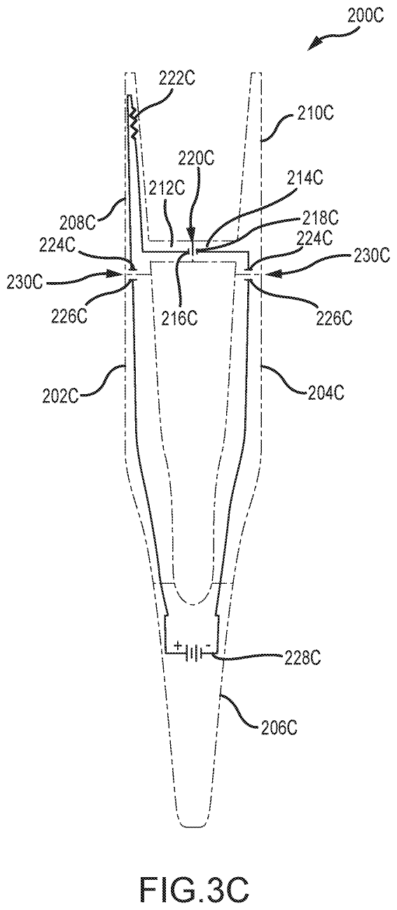

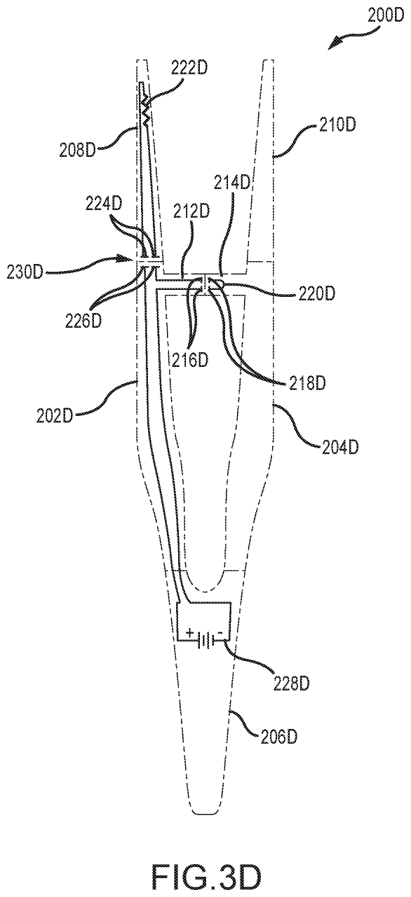

FIG. 3A is a schematic diagram of an electronic circuit 200A utilized in a gripping instrument. The portions of the circuit 200A are disposed in the parts of the gripping instrument, as described elsewhere herein. The portions of the gripping instrument are depicted schematically, as a first arm 202A, a second arm 204A, a handle 206A, a first arm gripping portion 208A, and a second arm gripping portion 210A. Each gripping portion 208A, 210A includes stops 212A, 214A that project therefrom and include one or more contacts 216A, 218A that are incorporated into the electronic circuit 200A. In this and the following figures, the stops are depicted schematically to depict location of the electrical circuits and contacts. The contacts 216A, 218A form a contact switch 220A that is used to deliver electricity to the cutting element 222A, depicted generally as a resistor. Since the gripping portion 208A is disconnectable from the first arm 202A, contacts 224A, 226A are utilized to connect a battery 228A disposed in the handle 206A to the remainder of the circuit 200A. This connection at an arm/grip interface 230A may include a mechanical or other interlock to ensure the gripping portion 208A remains engaged with the arm 202A during use of the instrument. The battery 228A may be removable or rechargeable. In examples, the battery 228A is an onboard battery 228A that may be recharged by plugging the instrument into a power source, via a removable power cable, USB cable, other cable, base charging station, or other component. The battery 228A is typically disposed in the handle 206A because the handle 206A may provide a greater volume of material to contain the battery 228A. Additionally, disposing the battery 228A in the handle 206 may help balance the weight of the battery 228A within the instrument and thus, the instrument in the hand. Other configurations of circuits are contemplated. For example, a cutting element 222A may be disposed on the second gripping portion. The electrical circuit for such a configuration may be a mirror image of that depicted in FIG. 3A, but with the circuit being disposed substantially in the second arm 204A. Other configurations are depicted in the following figures.

For example, FIG. 3B depicts another circuit 200B configuration. A number of the components depicted in FIG. 3B are described above with regard to FIG. 3A and are therefore not necessarily described further. In the depicted circuit 200B a portion of the circuit 200B is routed through the second arm 204B. Contacts 224B, 226B at the arm/grip interface 230B connect so as to at least partially complete the electronic circuit 200B. A stop 212B contacts a deformable button 214B that forces a contact switch 220B to close, thus completing the circuit 200B. This activates the cutting element 222B that is, in this case, disposed on the second gripping portion 210B.

FIG. 3C depicts another circuit 200C configuration. A number of the components depicted in FIG. 3C are described above with regard to FIG. 3A and are therefore not necessarily described further. In the depicted circuit 200C, a portion of the circuit is routed through both the first arm 202C and the second arm 204C. Contacts 224C, 226C at each arm/grip interface 230C connect to as to at least partially complete the electronic circuit 200C. Stops 212C, 214C each include one contact 216C, 218C that act as a contact switch 220C when in contact, which activates the cutting element 222C that is, in this case, disposed on the first gripping portion 208C. The depicted configuration of the circuit 200C may be desirable because of the symmetrical configuration of the circuit 200C within the handle 206C, first arm 202C, and second arm 204C. This allows for disposition of the cutting element 222C on either or both of the first gripping portion 208C and the second gripping portion 210C as required or desired for particular applications. FIG. 3D is a schematic diagram of an electronic circuit 200D utilized in a gripping instrument. A number of the components depicted in FIG. 3D are described above with regard to FIG. 3A and are therefore not necessarily described further. Here, the contacts 216D, 218D are disposed in stops 212D, 214D that form a portion of the first and second arms 202D, 204D.

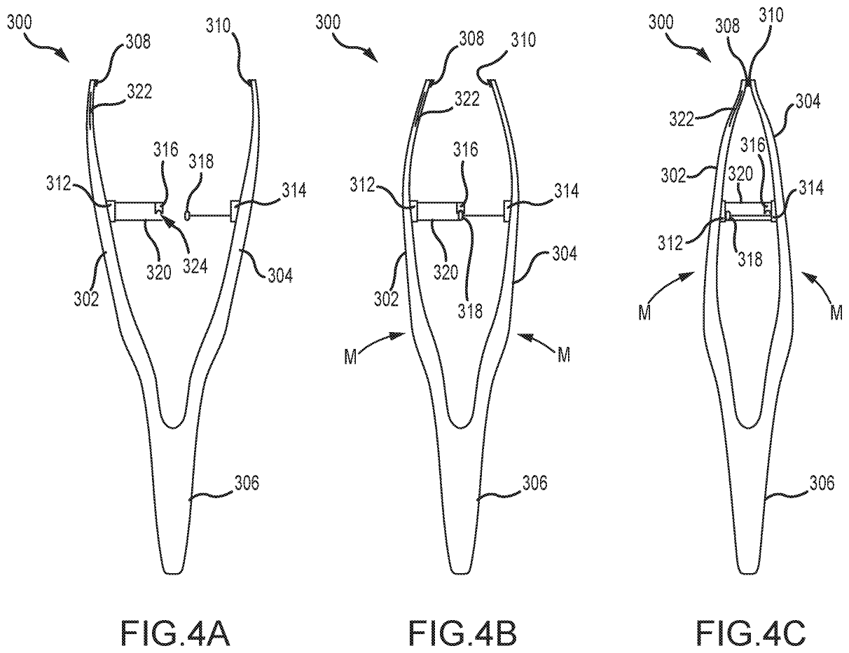

FIGS. 4A-4C are enlarged schematic views of a gripping instrument 300 in a plurality of positions. The gripping instrument 300 includes a first arm 302 and a second arm 304 joined at a handle 306. Each of the first arm 302 and the second arm 304 include a tip 308, 310 that is used to grip an object such as sutures. Each arm 302, 304 also includes a stop 312, 314 having a contact 316, 318 thereon. A sleeve 320 forms a part of the stop 312 and includes the contact 316. A cutting element 322 is disposed on the first arm 302 and, in this configuration, each arm 302, 304 is a single piece (e.g., without a disconnectable gripping portion as depicted elsewhere herein). In FIGS. 4A-4C, the contacts 316, 318 are depicted schematically for clarity so as to explain the operation of the gripping instrument 300. The electronic circuit is not depicted but configurations thereof will be apparent to a person of skill in the art.

In the first position depicted in FIG. 4A, the gripping instrument 300 is positioned such that both arms 302, 304 are disposed away from each other. In this configuration, the tips 308, 310 are not in contact and the gripping instrument 300 is not gripping an object such as sutures. Additionally, the contacts 316, 318 are disposed away from each other and, as such, the cutting element 322 is not activated. In FIG. 4B, the arms 302, 304 have been moved M closer to each other. As such, the contacts 316, 318 complete the electronic circuit and activate the cutting element 322. To ensure contact between the contacts 316, 318, a sleeve or other structure 320 is configured to receive contact 318. Additionally, at least one contact 316, 318 may form a detent 324 into which the other contact 316, 318 engages so as to help position the contacts 316, 318 in the engaged position. Other receiving or guidance structures may be utilized. In this second position, the tips 308, 310 are still not in contact, so gripping of an object is not possible. In this position, the cutting element 322 may be utilized to cut a suture. In FIG. 4C, movement M continues until the tips 308, 310 are in contact. As such, in this position, the tips 308, 310 can grip objects (such as sutures that were previously cut with the cutting elements).

The instrument 300 is typically held in a first orientation, where the thumb is in contact with a first arm 302, while the index finger is in contact with the second arm 304. A user, e.g., a doctor, may actuate the gripping action of the instrument 300 to grip and pull sutures being used to close a wound. At the end of the suturing operation, the cutting element 322 may be used to melt through the polypropylene suture so as to cut the suture to the proper length, without using the gripping function of the instrument 300, and without changing the first orientation of the instrument 300. The melting/cutting action can be selectively performed, e.g., by selectively placing the cutting element 322 proximate the suture, squeezing the instrument 300 until the contacts 316, 318 engage, then touching the suture to the cutting element 322. Further squeezing of the arms 302, 304 brings the tips 308, 310 closer together so as to pinch or grasp the melted/cut suture therebetween. As such, the instrument 300 with cutting element 322 is very easy to use and the cutting element can be selectively actionable based on a position of the forceps (e.g., the arms thereof).

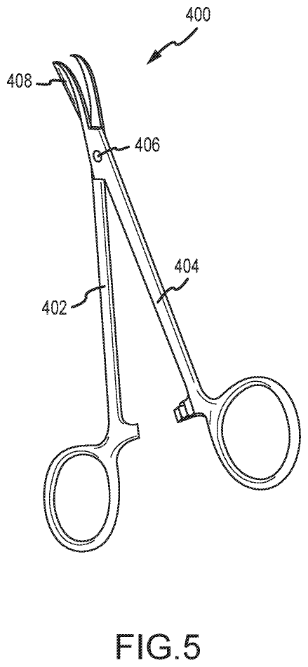

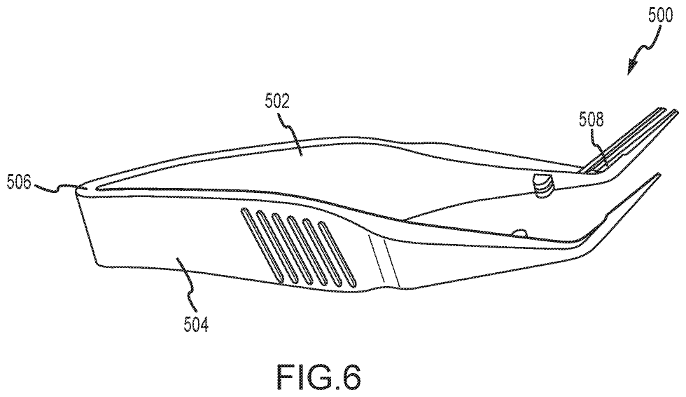

In the embodiments depicted above, the gripping instruments have one or more cutting elements attached thereto. As noted, these gripping instruments include two arms joined via a weld or adhesive at the joint. The incorporation of blades into other types of gripping instruments is also contemplated, however. For example, FIG. 5 depicts a gripping instrument 400 having two arms 402, 404 joined at a joint 406. The joint 406 is, in this case, a pivoting connection such as a rivet, screw, or a bolt. As with the other examples depicted herein, a cutting element 408 can be integrated into one or more of the arms 402, 404, and utilized similarly to the examples described above. FIG. 6 depicts a gripping instrument 500 having two arms 502, 504 joined at a joint 506 that in this case, is a live hinge. As with the other examples depicted herein, a cutting element 508 can be integrated into one or more of the arms 502, 504, and utilized similarly to the examples described above.

In examples, the battery may be a rechargeable battery having a voltage. The cutting element may be a wire having a gauge. A breaker may be incorporated into the circuit such that overheating of the cutting element trips the breaker to prevent damage to the circuit. Other electronic components such as capacitors, resistors, etc., may be incorporated a required or desired for a particular application. Other switches (e.g., toggle switches or user-actuated buttons) may be utilized.

The materials utilized in the manufacture of the instrument may be those typically utilized for other equipment utilized in surgical suites. For instruments or portions of instruments (e.g., handles) that may be reused, metals such as steel, aluminum, or stainless steel can be used. Additionally, robust reinforced plastics can also be used. Such materials utilized in reusable instruments should be able to withstand temperatures generated in an autoclave. Disposable plastics can also be utilized. It may be desirable, however that these materials could also be sterilized prior to use.

The terms first and second, as used herein, are relative terms used for convenience of the reader and to differentiate various elements of the instruments from each other. In general, unless otherwise noted, the terms are not meant to define or otherwise restrict location of any particular element.

While there have been described herein what are to be considered exemplary and preferred examples of the present technology, other modifications of the technology will become apparent to those skilled in the art from the teachings herein. The particular methods of manufacture and geometries disclosed herein are exemplary in nature and are not to be considered limiting. It is therefore desired to be secured in the appended claims all such modifications as fall within the spirit and scope of the technology. Accordingly, what is desired to be secured by Letters Patent is the technology as defined and differentiated in the following claims, and all equivalents.

What is claimed is:

1. An apparatus comprising:
a first arm having a first gripping end and a first handle end;
a second arm having a second gripping end and a second handle end, wherein the first handle end and the second handle end are joined so as to form a handle;
a cutting element disposed on the first arm, proximate the first gripping end; and
an electronic circuit at least partially disposed in the first arm, wherein the electronic circuit is connected to the cutting element, and wherein the electronic circuit is selectively activatable so as to deliver an electric signal to the cutting element.

2. The apparatus of claim 1, further comprising a switch connected to the electronic circuit, wherein the switch selectively activates the electronic circuit.

3. The apparatus of claim 2, wherein the switch comprises a first contact on the first arm and a second contact on the second arm,
wherein when the first arm is in a first position, the switch is in a non-activated configuration, and
wherein when the first arm is in a second position, the switch is in an activated configuration.

4. The apparatus of claim 3, wherein when in the first position, the first gripping end and the second gripping end are not in contact, and wherein when in the second position, the first gripping end and the second gripping end are not in contact.

5. The apparatus of claim 4, wherein when the first arm is in a third position, the switch is in a non-actuated position and the first gripping end and the second gripping end are in contact.

6. The apparatus of claim 5, wherein the second position is disposed between the first position and the third position.

7. The apparatus of claim 1, further comprising a battery disposed in the handle.

8. The apparatus of claim 1, wherein the first gripping end is detachably engaged with the first handle end, and wherein the second gripping end is detachably engaged with the second handle end.

9. An apparatus comprising:
a forceps; and
a cutting element connected to at least one arm of the forceps, wherein the cutting element is selectively activatable based on a position of the forceps.

10. The apparatus of claim 9, further comprising:
a contact disposed on each arm of the forceps; and
an electronic circuit disposed in the forceps, wherein the electronic circuit is connected to the cutting element and the contacts.

11. The apparatus of claim 10, wherein when the forceps are in a first position, the electronic circuit is open.

12. The apparatus of claim 11, wherein when the forceps are in a second position, the electronic circuit is closed.

13. The apparatus of claim 12, wherein when the forceps are in a third position, the electronic circuit is open, and wherein the second position is between the first position and the third position.

14. The apparatus of claim 9, further comprising a rechargeable battery in communication with the electronic circuit.

15. The apparatus of claim 6, wherein the at least one arm is disconnectable from the forceps.

16. An apparatus comprising:
a forceps having a first arm, a second arm, and a handle portion connecting the first arm and the second arm, wherein each of the first arm and the second arm terminates at a connector element;
a first gripping element configured to be releasably connected to the first arm connector element;
a second gripping element configured to be releasably connected to the second arm connector element;
an electronic circuit at least partially disposed in the first arm; and
a cutting element disposed on the first gripping element, wherein the cutting element is configured to electronically engage with the electronic circuit.

17. The apparatus of claim 16, further comprising a control switch operatively coupled to the electronic circuit.

18. The apparatus of claim 17, wherein the control switch comprises a first contact on at least one of the first arm and the first gripping element and a second contact on at least one of the second arm and the second gripping element.

19. The apparatus of claim 18, further comprising at least one of a battery and a power cord connected to the electronic circuit.

20. The apparatus of claim 16, wherein the first connector arm comprises a cutting element contact in communication with the electronic circuit.
